(19) United States
(12) Patent Application Publication
(10) Pub. No.: US 2006/0009773 A1
(43) Pub. Date: Jan. 12, 2006

(54) Helical interlocking mating guide and advancement structure

(76) Inventor: Roger P. Jackson, Prairie Village, KS (US)

Correspondence Address: LAW OFFICE OF JOHN C. MCMAHON, P.O. BOX 30069, KANSAS CITY, MO, 64112, US

(21) Appl. No.: 11/227,929

(22) Filed: Sep. 15, 2005

Related U.S. Application Data
(63) Continuation-in-part of application No. 10/831,919, filed on Apr. 26, 2004.
Application No. 10/831,919 is a continuation-in-part of application No. 10/236,123, filed on Sep. 6, 2002, now Pat. No. 6,726,689.

(51) Int. Cl. A61B 17/56

(52) U.S. Cl. 606/73

(57) ABSTRACT

A medical implant structure includes a pair of helically wound interlocking forms located on a cylindrical closure for an open headed medical implant and in a receiver between arms of the implant respectively. The interlocking forms each include overlapping gripping elements that engage mating elements during assembly to prevent radial splaying of the arms of the implant. The structure includes dove-tail-like and jig-saw-puzzle-like interlocking forms.

### CROSS-REFERENCE TO RELATED APPLICATIONS

This is a continuation-in-part of co-pending U.S. patent application Ser. No. 10/831,919 filed Apr. 26, 2004 which is a continuation-in-part of U.S. patent application Ser. No. 10/236,123 filed Sep. 6, 2002, now U.S. Pat. No. 6,726,689.

### BACKGROUND OF THE INVENTION

The present invention is directed to a closure for use between spaced arms of a medical implant for securing a rod to the implant. The structure includes a first interlocking form on a closure and a mating second interlocking form on a receiver. The closure is operably rotated into the receiver. The first and second interlocking forms are both helically wound so that the first interlocking form advances relative to the second interlocking form, when the closure with the first interlocking form is inserted in the receiver and rotated. At least one of the first or second interlocking forms includes a projection that overlaps and radially locks with the other interlocking form when the two forms are mated.

Medical implants present a number of problems to both surgeons installing implants and to engineers designing them. It is always desirable to have an implant that is strong and unlikely to fail or break during usage. It is also desirable for the implant to be as small and lightweight as possible so that it is less intrusive on the patient. These are normally conflicting goals, and often difficult to resolve.

One particular type of implant presents special problems. In particular, spinal bone screws, hooks, etc. are used in many types of back surgery for repair of injury, disease or congenital defect. For example, spinal bone screws of this type are designed to have one end that inserts threadably into a vertebra and a head at an opposite end thereof. The head is designed to receive a rod or rod-like member, a chord, ligament or other type of metal or non-metal longitudinal connecting member in a channel in the head, which longitudinal member is then both captured in the channel and locked in the head to prevent relative movement between the various elements subsequent to installation.

There are two different major types of bone screws and similar devices which are classified as closed headed and open headed. While the closed headed devices are highly effective at capturing and securing a rod, since the rod is threaded through an opening in the head, it is very difficult during surgery to thread the rod through the heads. This is because there are many heads and the rod is curved or the heads do not align. Consequently, the more screw heads that the rod must pass through, the more difficult it is to thread the rod into them.

The second type of head is an open head wherein a channel is formed in the head and the rod or longitudinal connecting member is simply laid in an open channel. The channel is then closed with a closure member. The open headed bone screws and related devices are much easier to use and in some situations must be used instead of the closed headed devices.

While the open headed devices are often necessary and often preferred for usage, there is a significant problem associated with them. In particular, the open headed devices conventionally have two upstanding arms that are on opposite sides of a channel that receives the rod member. The top of the channel is closed by a closure member after the rod member is placed in the channel. The closure can be of a slide in type, but such are not easy to use. Threaded nuts are sometimes used that go around the outside of the arms. Such nuts prevent splaying of the arms, but nuts substantially increase the size and profile of the implant which is not desirable. Many open headed implants are closed by plugs that screw into threads between the arms, because such have a low profile. However, threaded plugs have encountered problems also in that they produce radially outward forces that lead to splaying of the arms or at least do not prevent splaying that in turn loosens the implant. In particular, in order to lock the rod member in place, a significant force must be exerted on the relatively small plug or screw. The forces are required to provide enough torque to insure that the rod member is clamped or locked in place relative to the bone screw, so that the rod does not move axially or rotationally therein. This typically requires torques on the order of 100 inch-pounds.

Because open headed implants such as bone screws, hooks and the like are relatively small, the arms that extend upwardly at the head can be easily bent by radially outward directed forces due to the application of substantial forces required to lock the rod member. Historically, early closures were simple plugs that were threaded with V-shaped threads and which screwed into mating threads on the inside of each of the arms. But, as noted above, conventionally V-shaped threaded plugs tend to splay or push the arms radially outward upon the application of a significant amount of torque, which ends up bending the arms sufficiently to allow the threads to loosen or disengage and the closure to fail. To counter this, various engineering techniques were applied to allow the head to resist the spreading force. For example, the arms were significantly strengthened by increasing the width of the arms by many times. This had the unfortunate effect of substantially increasing the weight and the profile of the implant, which was undesirable.

Many prior art devices have also attempted to provide outside rings or some other type of structure that goes about the outside of the arms to better hold the arms in place while the center plug is installed and thereafter. This additional structure may cause the locking strength of the plug against the rod to be reduced which is undesirable, especially when the additional structure is partly located beneath the plug. Also, the additional elements are unfavorable from a point of view of implants, since it is typically desirable to maintain the number of parts associated with the implants at a minimum and, as noted above, the profile as minimal as possible.

Other designers have attempted to resolve the splaying problem by providing a closure with a pair of opposed radially extending wedges or flanges that have mating structure in the arms of the implant. Such devices serve as a closure and do somewhat resist splaying of the arms, but are often very difficult to use. In particular, the rods normally have some curvature as the rods are bent to follow the curvature of the spine and normally bow relative to the bottom of the bone screw channel that receives such a rod. The rod thus fills much of the channel and must be “unbent” to rest on the bottom of the channel and be held securely in place. Therefore, the rod is preferably compressed by the plug and unbent by advancement of the plug into the channel in order to assume that the plug will securely hold the rod and that the rod and plug will not loosen when post assembly forces are placed on the rod. Because it takes substantial force to unbend the rod, it is difficult to both place the plug fully in the channel and rotate it for locking while also trying to line up the wedges with the mating structure. It is much easier to align the plug mating structure with the mating structure of the arms at the top of the arms and then rotate the plug so as to screw the plug into a plug receiver to advance the plug toward the rod. In this way the plug starts applying significant force against the rod only after parts of the mating structure have at least partly joined at which time torque can be applied without having to worry about alignment. It is noted that where wedges are used, the cross-section of the structure changes therealong so that the device “locks up” and cannot turn further after only a small amount of turning, normally ninety degrees.

Consequently, a lightweight and low profile closure plug was desired that resists splaying or spreading of the arms while not requiring significant increases in the size of the screw or plug heads and not requiring additional elements that encircle the arms to hold the arms in place.

It is noted that the tendency of the open headed bone screw to splay is a result of the geometry or contour of the threads typically employed in such devices. In the past, most bone screw head receptacles and screw plugs have employed V-shaped threads. V-threads have leading and trailing sides oriented at angles to the screw axis. Thus, torque on the plug is translated to the bone screw head at least partially in an axial direction, tending to push or splay the arms of the bone screw head outward in a radial direction. This in turn spreads the internally threaded receptacle away from the thread axis so as to loosen the plug in the receptacle.

The radial expansion problem of V-threads has been recognized in various types of threaded joints. To overcome this problem, so-called “buttress” threadforms were developed. In a buttress thread, the trailing or thrust surface is oriented substantially perpendicular to the thread axis, while the leading or clearance surface remains angled. This theoretically results in a neutral radial reaction of a threaded receptacle to torque on the threaded member received.

Development of threadforms proceeded from buttress and square threadforms which in theory have a neutral radial effect on the screw receptacle to reverse angled threadforms which theoretically positively draw the threads of the receptacle radially inward toward the thread axis when the plug is torqued. In a reverse angle threadform, the trailing side of the external thread is angled toward the thread axis instead of away from the thread axis, as in conventional V-threads. While buttress, square and reverse threadforms reduce the tendency to splay, the arms can still be bent outward by forces acting on the implant and the threads can be bent by forces exerted during installation. Therefore, while certain threadforms may not exert radial forces during installation, at most such threadforms provide an interference or frictional fit and do not positively lock the arms in place relative to the closure plug.

Finally, it is noted that plugs of this type that use threadforms are often cross threaded. That is, as the surgeon tries to start the threaded plug into the threaded receiver, the thread on the plug is inadvertently started in the wrong turn or pass of the thread on the receiver. This problem especially occurs because the parts are very small and hard to handle. When cross threading occurs, the plug will often screw part way in the receiver and then “lock up” so that the surgeon is led to believe that the plug is properly set. However, the rod is not tight and the implant fails to function properly. Therefore, it is also desirable to have a closure that resists crossthreading in the receiver.

### SUMMARY OF THE INVENTION

A non threaded guide and advancement structure is provided for securing a set screw, plug or closure in a receiver. Preferably the receiver is a rod receiving channel in an open headed bone screw, hook or other medical implant wherein the channel has an open top and is located between two spaced arms of the implant.

The guide and advancement structure has a first part or interlocking form located on the closure and a second part or interlocking form that is located on the interior of the receiving channel.

Both parts of the guide and advancement structure are spirally or more preferably helically wound and extend about the closure and receiving channel for at least one complete 360° pass or turn. Preferably, both parts include multiple turns such as 2 to 4 complete 360° rotations about the helixes formed by the parts. The helixes formed by the parts are coaxial with the closure when the closure is fully received in or being rotated into the receiving channel between the arms.

One major distinguishing feature of the guide and advancement structure is that each of the parts include elements that mechanically interlock with the opposite part as the closure is rotated and thereby advanced into the receiving channel toward the bottom of the channel and into engagement with a rod received in the channel.

Each part of the guide and advancement structure preferably has a generally constant and uniform cross-section, when viewed in any cross-sectional plane fully passing through the axis of rotation of the closure during insertion, with such uniform cross-section extending along substantially the entire length of the interlocking form. It is noted that at opposite ends of each interlocking form, the form must be feathered or the like and so the cross-section does change some at such locations, while retaining part of the overall shape. In particular, the outer surfaces of each interlocking form remain sufficiently uniform to allow interlocking forms to be rotated together and slide tangentially with respect to each other through one or more complete turns of the closure relative to the receiving channel. Each part may be continuous from near a bottom of the closure or receiving channel to the top thereof respectively. In certain circumstances one or both parts may be partly discontinuous, while retaining an overall helical configuration with a generally uniform cross-sectional shape. When the interlocking form has multiple sections due to being discontinuous, each of the sections has a substantially uniform cross-section along substantially the entire length thereof.

In order to provide an interlocking structure, the parts of the structure include helical wound projections or interlocking forms that extend radially outward from the closure and radially inward from the receiving channel. The interlocking forms may be of many different shapes when viewed in cross-section with respect to a plane passing through the axis of rotation of the plug during insertion. In general, the interlocking forms increase in axial aligned width or have a depression at a location spaced radially outward from where the interlocking form attaches to a respective closure or receiving channel, either upward (that is, parallel to the axis of rotation of the closure in the direction from which the closure comes or initially starts) or downward or in both directions. This produces a first mating element that is in the form of a protrusion, bump, lip, ridge, elevation or depression on the interlocking form that has a gripping or overlapping portion. The opposite interlocking form has a second mating element with an interlocking gripping or overlapping portion that generally surrounds or passes around at least part of the first mating element in such a way that the two are radially mechanically locked together when the closure is advanced into the receiving channel.

Therefore, in accordance with the invention a mating and advancement structure is provided for joining two devices, that are preferably medical implants and especially are an open headed implant that includes a rod or longitudinal connecting member receiving channel and a closure for closing the receiving channel after the rod is received therein. The mating and advancement structure includes a pair of mateable and helical wound mechanical interlocking forms with a first interlocking form located on an outer surface of the closure and a second interlocking form located on an inner surface of the receiving channel or receiver. The first and second interlocking forms are startable so as to mate and thereafter rotatable relative to each other about a common axis so as to provide for advancement of the closure into the receiver during assembly when the closure interlocking form is rotated into the receiver interlocking form. The first and second interlocking forms have a helical wound projection that extends radially from the closure and the receiver respectively. Each interlocking form projection has a base that is attached to the closure or receiver respectively and preferably includes multiple turns that may each be continuous or partially discontinuous with constant or uniform cross-sectional shape. The interlocking forms have substantial axial width near an outer end thereof that prevents or resists misalignment of the interlocking form during initial engagement and rotation thereof.

After assembly, in some embodiments each turn of each projection generally snugly engages turns of the other projection on either side thereof. In other embodiments there must be sufficient tolerances for the parts to slide tangentially, so that when thrust surfaces of the interlocking forms are very close during tightening, some gap occurs on the leading side of the closure interlocking form. In such a case the portions of the interlocking forms on the thrust side thereof lock together and prevent radial splaying. Located radially spaced from where the base of each projection is attached to either the closure or receiver respectively, is an axially extending (that is extending in the direction of the axis of rotation of the plug or vertically) extension or depression. The opposite or mating interlocking form has elements that wrap around or into such extensions or depressions of the other interlocking form. That is, the forms axially inter-digitate with each other and block radial movement, expansion or splaying. In this way and in combination with the interlocking forms preferably being snug relative to each other with sufficient clearance to allow rotation, the interlocking forms, once assembled or mated lock to prevent radially slipping or sliding relative to each other, even if the base of one or both is bent relative to the device upon which it is mounted. It is possible that the cross-section of the projection (in a plane that passes through the plug axis of rotation of the plug) of each section of each turn or pass of the interlocking form be the same, although this is not required in all embodiments. For example, part of the interlocking form may be missing in the region between opposed arms when assembly is complete as this area is not required to hold the arms together.

The present invention provides a helically wound interlocking form for use in a medical implant closure which resists splaying tendencies of arms of a receiver. In one embodiment the interlocking form of the present invention provides a compound or “non-linear” surface on a trailing face, thrust face or loading flank of the interlocking form.

The interlocking form located on the closure in one embodiment is helically wound about a cylindrical outer surface of the closure and has an inner radius or root, and an outer radius or crest that remain constant over substantially the entire length of the interlocking form. The receiver has a mating or similar shaped interlocking form wound about the interior thereof. In this embodiment the interlocking form has leading or clearance surfaces and trailing or thrust surfaces, referenced to the direction of axial movement of the form when rotated into one another.

The structure also includes an internal helical wound interlocking form located on an internal surface of a receiver member and having an outer root and an inner crest. The internal interlocking form has thrust surfaces which are oriented in such a direction so as to be engaged by the thrust surfaces of the external interlocking form of a member engaged therewith.

In the interlocking forms of this series of embodiments, the thrust surfaces are “non-linear” or compound. That is, the thrust surfaces have a non-linear appearance when represented in cross-section. The purpose for the non-linear or compound surface is to provide a portion of the thrust surface which is oriented in such a direction as to resist a tendency of the receiver to expand or splay when tightening torque is applied to rotate the interlocking forms into a mating relationship. As applied to a closure for an open headed bone implant screw, the non-linear or compound surfaces of the interlocking forms resist splaying tendencies of the arms of the head. The objective of the interlocking form is not necessarily to generate a radially inwardly directed force on the receptacle in tightening the fastener (although this may occur in some embodiments), but more importantly to resist and prevent outward forces generated by engagement of the closure with the closure receptacle or by other forces applied to the components joined by the closure and closure receptacle. It is noted that the present invention requires that only a portion of the thrust surfaces of a closure be so configured as to face toward the closure axis and only a portion of thrust surfaces of a closure receptacle face away from the axis.

While the axial extension or depression in one series is located on the thrust or trailing surface, it is also possible for such to be located on the opposite or leading surface or both.

In this series of embodiments, a section of the interlocking form at the crest, that is located radially outward of the root, is enlarged in cross-sectional area to create a gripping, locking or stopping surface that resists slippage or sliding in a radial direction relative to an opposed interlocking form. In a complementary manner, a section of the interlocking form between the root and the crest and that is radially spaced from the root is enlarged in cross-sectional area to create a gripping, locking or stopping surface that engages a like surface of the opposite interlocking form. The enlarged sections of the inner and outer interlocking forms are created, in practice, by cutting, molding, machining or the like grooves or channels or the like into a radially inward portion of the thrust surface of the external interlocking form and mating grooves or channels into a radially outward portion of the thrust surface of the internal interlocking form. Such grooves or channels may be formed by specially shaped taps and dies, cutting elements or by other suitable manufacturing processes and technologies, including molding.

The interlocking forms of the present invention may be implemented in a variety of configurations of non-linear, compound, or complex trailing and/or leading surfaces. The nomenclature used to describe variations in the interlocking forms of the present invention is especially referenced to the external interlocking forms located on a closure, with complementary or similar shapes applied to the internal interlocking forms on a receiver. In an axial shoulder interlocking form of the present invention, a somewhat squared gripping shoulder is formed on an outer periphery of the external interlocking forms and an inner gripping surface on the internal interlocking forms. The axial shoulder interlocking form results in complementary cylindrical surfaces on the external and internal interlocking forms which mutually engage when the fastener or closure is rotated into a closure receptacle.

In an axial extending bead interlocking form, the external interlocking form is provided with a rounded peripheral bead or lateral lip which projects in an axial direction along the interlocking form crest and a complementary rounded concave channel in the internal interlocking form. The reverse occurs with the internal interlocking form.

In a radial bead interlocking form, a rounded bead enlargement is formed on the radially outward periphery at the crest of the external interlocking form, while the internal interlocking form is formed in a complementary manner to receive the radial bead interlocking form.

A scalloped or scooped interlocking form is, in effect, a reciprocal of the axial bead interlocking form and has a rounded channel or groove located along the thrust surface of the external interlocking form, with a complementary rounded convex bead shape formed associated with the internal interlocking form.

A variation of the axial bead interlocking form is a medial bead embodiment. In the medial bead interlocking form, a bead projects from a base thrust surface of an external interlocking form in an axial direction at a location medially between the root and crest of the interlocking form. In a complementary medial bead internal interlocking form, an axial groove is formed in a base thrust surface between the root and crest. In a medial groove interlocking form, an axial groove is formed in a base thrust surface of the external interlocking form medially between the root and crest, while the internal interlocking form has an axial bead located medially between the root and crest.

Variations in the above described interlocking forms are envisioned with respect to relative extensions or enlargements and depressions or depth of grooves of the various interlocking forms. In some variations, the opposite interlocking forms have the same but reversed and inverted cross-section, whereas in others the cross-section of the paired interlocking forms is different. It is noted that many other configurations of interlocking forms with non-linear, compound or complex thrust surfaces are envisioned, which would be encompassed by the present invention.

The interlocking forms of the present invention find particularly advantageous application in various types of bone implant devices, although the inventive interlocking forms are not limited to such use. The interlocking forms also have advantages in reducing misalignment problems of cross-interlocking and mis-interlocking of interlocking forms when the opposed interlocking forms are joined and rotated which is commonly encountered in such devices when threads of various types are used.

### OBJECTS AND ADVANTAGES OF THE INVENTION

Therefore, objects of the present invention include: providing an improved closure for an open headed lightweight and low profile medical implant wherein the implant has a pair of spaced arms and the closure closes between the arms; providing such a closure which includes a pair of opposed interlocking forms and which resists tendencies of the arms to splay or separate during insertion of the closure, to thereby reduce the likelihood of failure of the implant and closure system during use; providing such a closure which can be installed at comparatively high torques to thereby secure the closure in the receiver channel and in certain embodiments to also lock a rod member in the open head of the implant where the closure engages and is urged against the rod by rotation in a receiver channel of the remainder of the implant; providing an interlocking form for such a closure which resists tendencies of parts of the channel receiver to expand radially outward in response to high torque applied to the closure; providing such an interlocking form in which the respective thrust surfaces of mating internal and external interlocking forms are “non-linear”, compound, or complex to provide only a portion of each trailing or leading surface which is oriented in such a direction as to resist the splaying or expanding tendencies of parts of the receiving channel; providing such an interlocking form wherein the interlocking form has a base that is secured to a member and the interlocking form extends radially outward from the base with an axial extension starting at or radially spaced from the base and further wherein the interlocking form has an extension or depression that extends in an axial direction relative to an axis of rotation of the interlocking form and which mates with the opposite interlocking form so as to grip or hold such extension or depression and yet further wherein opposed interlocking forms are rotatable relative to each other during assembly, but are preferably sufficiently snug or located sufficiently near to one another to prevent one interlocking member to slide radially past another when torque is applied thereto or when forces act on the implant; providing embodiments of such an interlocking form having an enlarged radial cross-section wherein the enlargement is spaced radially outward of a root of the external interlocking form and a complementary enlarged cross-section spaced radially inward of a root of the internal interlocking form; providing embodiments of such an interlocking form having a first groove or channel formed in a surface inward of a periphery of an external interlocking form and a complementary second groove or channel formed in a surface inward of a periphery of an internal interlocking form so that the paired interlocking forms overlap and radially lock together upon assembly; providing embodiments of such an interlocking form in which the enlarged peripheries and grooves of the external and internal interlocking form have or form angularly defined or axially extending shoulders; providing embodiments of such an interlocking form in which the enlarged peripheries of the external and internal interlocking form have or form arcuately defined or rounded shoulders; providing such interlocking forms having a generally uniform cross-section along a substantial length thereof; providing such interlocking forms that rotate relative to each other at least one full turn upon assembly; providing such interlocking forms which reduce the likelihood of cross-interlocking or mis-interlocking problems of members during initial joining; providing such interlocking forms which can be formed relatively economically using appropriate metal forming technologies; and providing interlocking forms, particularly for implant and bone fixation hardware, which are economical to manufacture, which are secure and efficient in use, and which are particularly well adapted for their intended usage.

Other objects and advantages of this invention will become apparent from the following description taken in conjunction with the accompanying drawings wherein are set forth, by way of illustration and example, certain embodiments of this invention.

The drawings constitute a part of this specification and include exemplary embodiments of the present invention and illustrate various objects and features thereof.

### BRIEF DESCRIPTION OF THE DRAWINGS

FIG. 1 is a perspective view of a closure for an open headed bone screw that has a helical wound gripping interlocking form in accordance with the present invention mounted thereon.

FIG. 2 is a side elevational view of the closure.

FIG. 3 is a side elevational view at a reduced scale and illustrates an interlocking form of the closure mated with and installed in a companion interlocking form on an open headed bone screw to capture a fixation rod within a head of the bone screw and with the head of the bone screw partially broken away to illustrate detail thereof.

FIG. 4 is an enlarged fragmentary side elevational view of the bone screw head with the closure installed therein, the closure and bone screw head incorporating the interlocking form according to the present invention with portions broken away to show detail thereof.

FIG. 5 is a view similar to FIG. 4 and illustrates details of first modified bone screw and closure showing a medial bead embodiment of an interlocking form of the present invention.

FIG. 6 is view similar to FIG. 4 and illustrates details of a second modified bone screw and closure showing an axial aligned shoulder embodiment of an interlocking form of the present invention.

FIG. 7 is a view similar to FIG. 4 and illustrates details of a third modified bone screw and closure showing an axial bead embodiment of an interlocking form of the present invention.

FIG. 8 is a view similar to FIG. 4 and illustrates details of a fourth modified bone screw and closure showing a shallow axial bead embodiment of an interlocking form of the present invention.

FIG. 9 is a view similar to FIG. 4 and illustrates details of a fifth modified bone screw and closure showing a radial bead embodiment of an interlocking form of the present invention.

FIG. 10 is a view similar to FIG. 4 and illustrates details of a sixth modified bone screw and closure showing a scalloped depression or scooped embodiment of an interlocking form of the present invention.

FIG. 11 is a fragmentary cross-sectional view of a seventh modified bone screw and closure, similar to the embodiment in FIG. 10, showing a pair of interlocking forms in accordance with the present invention.

FIG. 12 is a fragmentary cross-sectional view of an eighth modified embodiment of a bone screw and closure showing a pair of interlocking forms in accordance with the invention.

FIG. 13 is a fragmentary cross-sectional view of a ninth modified embodiment of a bone screw and closure showing a pair of interlocking forms in accordance with the invention.

FIG. 14 is a fragmentary cross-sectional view of a tenth modified embodiment of a bone screw and closure showing a pair of interlocking forms in accordance with the invention.

FIG. 15 is a view similar to FIG. 4 and illustrates details of an eleventh modified bone screw and closure showing a dove-tail or reversed wedge shaped embodiment of an interlocking form of the present invention.

FIG. 16 is a view similar to FIG. 4 and illustrates details of a twelfth modified bone screw and closure showing a radial bead embodiment of an interlocking form of the present invention, specifically of a jig-saw puzzle-type shape.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

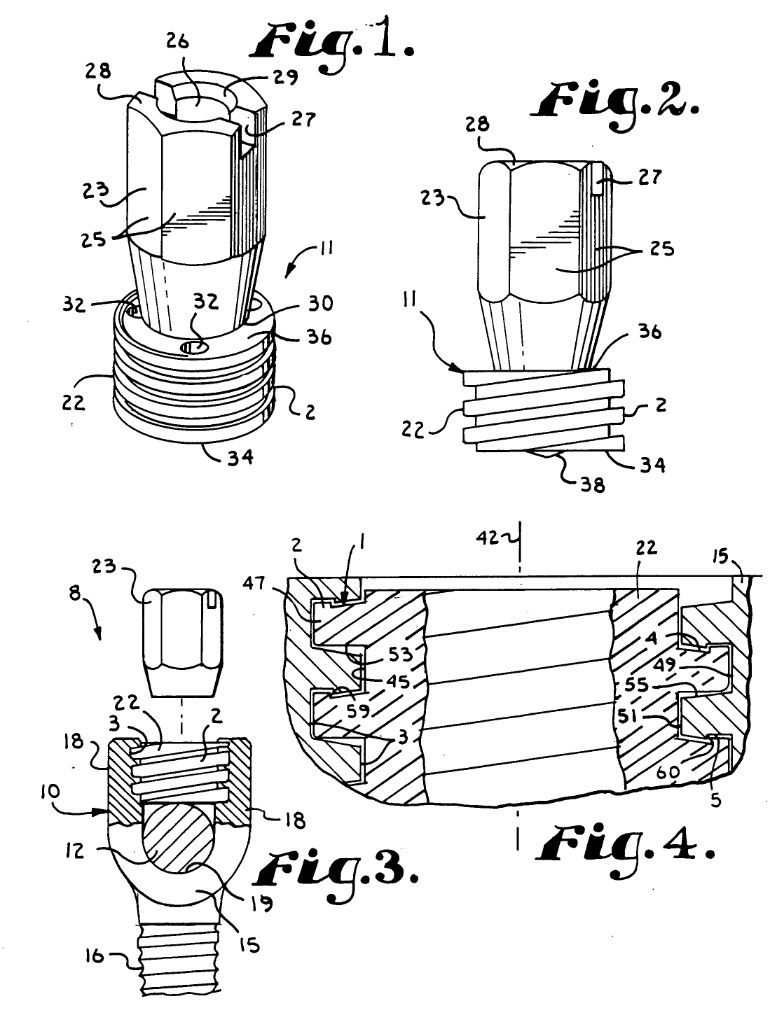

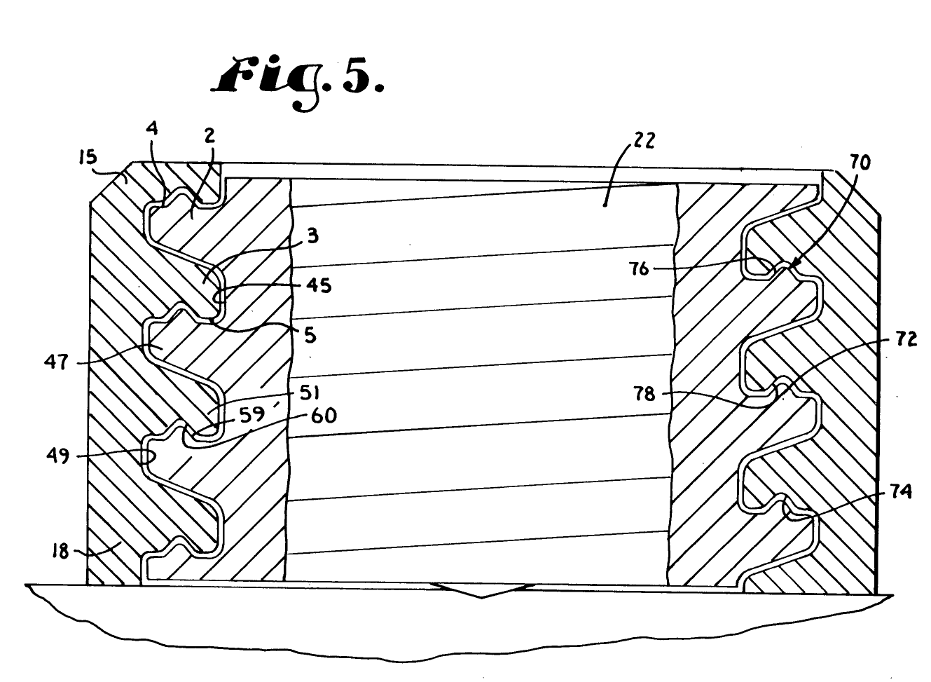

Referring to the drawings in more detail, the reference numeral 1 generally designates a gripping interlocking form arrangement incorporating a non-linear or compound surface which embodies the present invention. The interlocking form arrangement 1 includes an external interlocking form 2 and internal interlocking form 3 which have respective thrust surfaces 4 and 5 (FIG. 4) and which are used as pairs. The interlocking form arrangement 1 may be used on any of a number of interlocking formed devices, such as an implanted bone fixation system 8 (FIG. 3), including a receiver or open headed implant member 10 which receives a closure or closure member 11 (FIGS. 1 and 2) to secure a fixation member 12 therein. In the interlocking form arrangement 1 of the present embodiment, the thrust surfaces 4 and 5 are non-linear or compound in such a manner as to resist tendencies of the receptacle 10 to splay or expand when the closure member 11 is rotated therein.

The illustrated implant member 10 is also referred to as an open headed bone screw and includes a U-shaped implant receiver or head 15 and a threaded shank 16. The receiver or head 15 has a pair of spaced apart arms 18 forming a rod receiving channel 19. The arms 18 are radially inwardly tapped with the internal interlocking form 3 that is discontinuous between sides to receive the closure member 11. The illustrated shank 16 tapers to a point (not shown) and is externally threaded and adapted to be received in a bone, such as a vertebra, to anchor the rod 12 to such a bone.

The shank 16 and the receiver 15 may be attached in a variety of ways, such as the fixed or integral embodiment illustrated in FIG. 3. Alternatively, the shank may be swivelably attached to the receiver resulting in a polyaxial bone screw. For example, the shank may be attached to the receiver utilizing a spline capture connection described in U.S. Pat. No. 6,716,214 and incorporated by reference herein. In such a connection, the bone screw shank includes an upper end mateable with a retaining structure disposed within the receiver. The retaining structure includes a partially spherical surface that is slidingly mateable with a cooperating inner surface of the receiver, allowing for a wide range of pivotal movement between the shank and the receiver. As illustrated in U.S. Pat. No. 6,716,214, a closure structure biases a longitudinal connecting member or rod against the shank upper end which in turn biases the retaining structure into fixed frictional contact with the receiver, so as to fix the longitudinal connecting member or rod relative to the vertebra. In such polyaxial bone screws, the receiver and the shank cooperate in such a manner that the receiver and the shank can be secured at any of a plurality of angles, articulations or rotational alignments relative to one another and within a selected range of angles both from side to side and from front to rear, to enable flexible or articulated engagement of the receiver with the shank until both are locked or fixed relative to each other near the end of an implantation procedure. In addition to the spline connection illustrated in U.S. Pat. No. 6,716,214, other types of rod-on-bone screw shank polyaxial capture connections may be used including, but not limited to, threaded, conical, cylindrical and pinned connections, frictional connections, as well as integral downloadable and uploadable shanks, and the like. It is foreseen that helical interlocking guide and advancement structures according to the invention may also be utilized in polyaxial bone screws having receivers adapted to receive a compression member or insert between the bone screw shank and the rod.

It is also foreseen that implant members according to the invention may include receivers with spaced apart arms having main portions and elongate extensions or tabs to facilitate the capture and reduction of spinal fixation rods or other elongate members, after which the arm extensions or tabs are broken off at weakened areas to form a low profile implant. Such arm extensions would include a connected extension of the interlocking anti-splay components found on the inner surfaces of the main portions of the arms such that force can be applied to a closure and through the closure to a rod or other member positioned between the extensions without splaying the extensions, as the closure holds them in fixed position relative to each other and as the closure traverses between the extensions and then seamlessly into main portions of the arms, locking the rod in the bone screw head. Thereafter, the extensions or tabs may be broken off.

The illustrated closure member 11 includes a plug, base section or base 22 and a break off head section 23 that breaks from the base 22 at a preselected torque. It is foreseen that such a closure could be made without a break-off head and other structure could be added for torquing or removing the base section. Furthermore, it is foreseen that such a base both captures the rod and locks the rod as in the embodiment illustrated in FIGS. 1 to 4 or, alternatively, that the base could just capture the rod and a set screw could be used in a threaded bore in the base to lock the rod in place. The base section 22 is provided with the external interlocking form 2 which is compatible with the internal interlocking form 3 of the bone screw head 15. Both interlocking forms 2 and 3 are helically wound and rotatably mateable together through rotation or turning of the closure member 11 about a central axis 42 thereof. The head 23 includes structure for positive engagement by an installation tool (not shown) to install the closure member 11 in the bone screw member 10. The structure that allows for installation of the illustrated break off head 23 includes faces 25 forming a hexagonal shape or “hex” head to receive a complementary hexagonally shaped installation driver or tool. The head 23 also includes a central bore 26 and a cross bore slot 27. The outer end of the head 23 is chamfered at 28, and the bore 26 is provided with an interior conical countersink at 29. The region where the head 23 meets the base 22 is reduced in cross-sectional thickness to form a weakened breakaway or fracture region 30. The breakaway region 30 is designed so that the head 23 separates from the base 22 when a selected torque is applied by the installation tool, as is diagrammatically illustrated by breaking away of the head 23 in FIG. 3. The base 22 is preferably provided with structure to facilitate removal of the base 22 from the implant head 15, such as the illustrated removal bores 32. The bores 32 may be formed by drilling from a lower end surface 34 of the plug 22, since an upper end surface 36 of the plug 22 is normally not accessible for drilling the bores 32 prior to break-off of the head 23. It is foreseen that many different types of removal devices or structures can be utilized with the base such as: axially aligned bores with hex, torx or other multifaceted cross-section, step down bores for engagement by an easy out, bores at the periphery or non axially aligned on the face of the base, bores with a left handed thread or the like. Further, the same structure used to torque the base on installation may be used to remove the base.

The base 22 is rotated into the receiving member of the bone screw head 15 to clamp an elongate member, such as the fixation rod 12 therein for any of a variety of surgical purposes. In general, the rod 12 is used to fix the position of a bone or portion of a bone, such as a plurality of vertebrae. The rod 12 may be anchored relative to some vertebrae and, in turn, used to secure other vertebrae in desired positions or orientations or used to properly align a series of vertebrae. It is generally required that the union formed between the bone screw 10, closure 11 and the rod 12 be very tight or snug to avoid relative movement therebetween. The fixation system 8 preferably employs structure that positively engages and seats the head 15 and/or the base 22 with respect to the rod 12, such as a conical set point 38 formed on the bottom surface 34 of the base 22 which engages the rod 12. The point 38 positively “bites” into the surface of the rod 12 to help prevent rotational or axial movement of the rod 12 relative to the screw 10. Alternatively or in combination with a point 38, other structures may be used to positively engage the closure plug 22 with the rod 12, such as a sharp edged coaxial ring (not shown) having a V-shaped cross-section formed on the lower surface 34 of the base 22 or point extending upwardly from the channel.

The interlocking forms 2 and 3 are helical and are intended to advance the closure member 11 linearly along the axis of rotation 42 of the closure member 11 and the interlocking forms 2 and 3 relative to another member as the closure member 11 is rotated relative to the bone screw 10. A spatial reference for such rotation and linear movement is along the axis 42 (FIG. 4). The axis 42 locates the coincident axes of the external or radially outward interlocking form 2 of the base 22 and the internal or radially inward interlocking form 3 of the head 15, when the base 22 is inserted into the head 15 by starting at the top of the interlocking form 3 (top is up in FIG. 4) and rotated. The base 22 has a basic cylindrical shape, and the external interlocking form 2 includes a root 45 and a crest 47 formed by cutting a helical wound channel of the desired cross-section into the original surface of the base 22. The crest 47 of the external interlocking form 2 has a greater radius than the root 45. In a like manner, the internal interlocking form 3 of the head 15 of the screw 10 has a helical channel under cut there into, forming a root 49 and crest 51. The root 49 of the internal interlocking form 3 has a greater radius than the crest 51.

The thrust surfaces 4 and 5 respectively of the external and internal interlocking forms 2 and 3 engage frictionally when the base 22 is rotated into the head 15. The thrust surfaces 4 and 5 are located on the trailing sides respectively of the crests 47 and 51, as referenced to the tightening direction movement of the base 22 into the head 15. In general, there is minimal contact between the clearance surfaces 53 and 55 when the base 22 is rotated in a tightening direction into the screw head 15 to allow rotation. The clearance surfaces 53 and 55 may frictionally engage when the base 22 is rotated in a reverse direction to remove it from the screw head 15.

Frictional engagement of the thrust surfaces 4 and 5 due to rotation causes the base 22 to be advanced linearly along the axis 42 into the screw head 15. However, once the base 22 “bottoms out” by contact of the lower surface 34 or the set point 38 with the rod 12 and the rod 12 may be unbent and pushed downwardly as far as it will go into the channel or seat 19, further rotation of the base 22 cannot result in further linear movement of the base 22 within the head 15. The interlocking forms 2 and 3 thereafter are radially locked together and each turn or pass of the forms 2 and 3 is preferably sufficiently snug with respect to turns of the opposite interlocking form to prevent either form 2 or 3 from slipping or sliding radially past one another upon application of additional torque or with application of forces due to usage by the patient.

The various compound, complex, curvate, linear or non-linear interlocking form arrangements of the present invention are intended to resist splaying tendencies of the arms 18. In particular, each thrust surface 4 and 5 of the interlocking forms 2 and 3 have a gripping, blocking, overlapping or splay resisting surface 59 or 60 respectively which is oriented in such a direction as to resist splaying of the arms 18 of the screw head 15 when the base 22 is rotated to a high degree of torque. On the external interlocking form 2, the splay resisting surface 59 is directed generally toward or faces the axis 42. Conversely, on the internal interlocking form 3, the splay resisting surface 60 is directed generally away from or faces away from the axis 42. Each of the surfaces 59 and 60 in this manner wrap over or around the opposite and block substantial radially relative movement there between. It is especially noted that the surfaces 59 and 60 are extensions of the interlocking forms 2 and 3 in an axial direction (that is parallel to the axis 42 or up and down as seen in FIG. 4). This axial extension is spaced away from the juncture of the interlocking forms 2 and 3 with the base 22 and screw 10. It is foreseen that such an extension can take many shapes and configurations (some of which are shown herein) and may also functionally be depressions or grooves. In each case the paired interlocking forms, such as forms 2 and 3, overlap each other and are snug about each other so as to prevent substantial relative radial slippage or movement between them during and after assembly of the base 22 into the bone screw 10.

FIG. 5 illustrate a non-linear or compound thrust surface interlocking form arrangement 70 which is of a medial bead interlocking form type. The interlocking form arrangement 70 a thrust surface 4 located on a plug 22 and internal interlocking form 3 with thrust surfaces 5 within a head 15 of a bone screw 10. The thrust surfaces 4 and 5 are contoured to provide complementary, interacting, splay resisting surfaces 59 and 60 on the external and internal interlocking forms 2 and 3 respectively. The external interlocking form 2 is provided with a bead 72 on the thrust surface 4, and the internal interlocking form 3 is provided with a complementary channel or groove 74 formed into the thrust surface 5. The illustrated thrust surfaces 4 and 5 are substantially perpendicular to the axis 42; however, such surfaces may alternatively be angled somewhat with respect to the axis 42 so as to slope downward or upward as the surface extends radially outward.

The bead 72 is located at a radius which is between or medial with respect to the root 45 and crest 47 of the external interlocking form 2. Similarly, the groove 74 is located at a radius which is medial to the root 49 and crest 51 of the internal interlocking form 3. The illustrated bead 72 and groove 74 are rounded and somewhat triangular in cross-section. Alternatively, the bead and groove 72 and 74 could be pointed and triangular, squared off, or semicircular. It should also be noted that the bead and groove 72 and 74 could be replaced by a medial groove formed in the external interlocking form 2 on the thrust surface 4 and a medial bead formed on the thrust surface 5 of the internal interlocking form 3. An inwardly facing surface 76 of the bead 72 forms the splay resisting surface 59 thereof, while an outwardly facing surface 78 of the groove 74 forms the splay resisting surface of the groove 74. Engagement of the splay resisting surfaces 76 and 78, respectively of the bead 72 and groove 74, resists tendencies of the arms 18 of the screw head 15 to splay when the closure base 22 is rotated into the head 15.

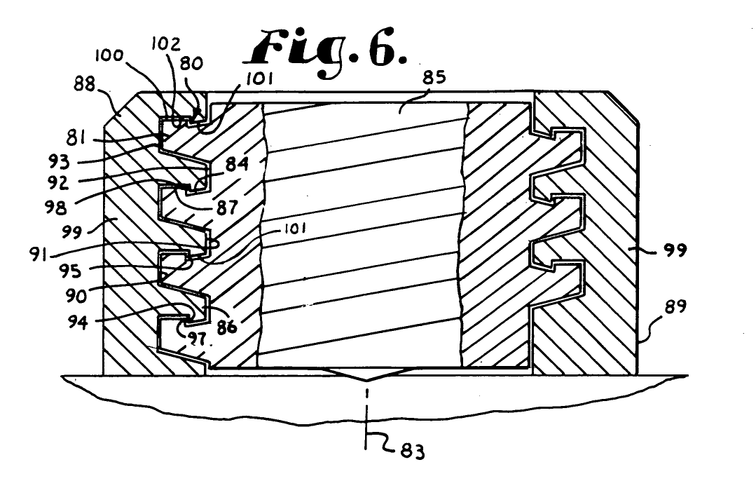

FIGS. 6 to 16 illustrate further variations in the paired interlocking forms of the present invention. In each case the base closure and bone screw, except as noted with respect to the interlocking forms, of the variations shown in FIGS. 6 to 14 are essentially the same as those shown in FIGS. 1 to 4, so only differing detail of the interlocking form structure will be described in detail and reference is made to the description given for FIGS. 1 to 4 for the remaining detail.

In FIG. 6, a guide and advancement structure 80 includes the external interlocking form 81 having an axially aligned shoulder or flange-like shaped configuration when view in cross-section in a plane passing through an axis of rotation 83. The interlocking form 81 has a thrust surface 84 on a base 85. The structure 80 also has an internal interlocking form 86 with a thrust surface 87 within the head 88 of a bone screw 89. The internal interlocking form 86 has a root 90 and a crest 91, while the external interlocking form 81 includes a root 92 and crest 93. The thrust surface 84 of the external interlocking form 81 includes an axially oriented or cylindrical shoulder 94 which forms a splay resisting surface 95 thereof.

Similarly, the thrust surface 87 of the internal interlocking form 86 includes a mating or complementary axially oriented or cylindrical shoulder 97 which forms a splay resisting surface 98. Engagement of the splay resisting surfaces 95 and 98 resists tendencies of the arms 99 of the head 88 to splay when the plug or base 85 is rotated into the head 88 and torqued tightly or at later times during usage. It is foreseen that a variation of the axial shoulder interlocking form would provide shoulders at inclined angles (not shown) to the axis 83. The illustrated splay resisting shoulder 94 is formed by a rectangular cross-section bead 100 formed on the thrust surface 84 of the external interlocking form 81. Similarly, splay resisting shoulder 97 is formed by a somewhat rectangularly cross-section shaped bead or foot portion 101 adjacent a groove 102 for receiving bead 100 and formed in the thrust surface 87 of the internal interlocking form 86. The interlocking forms 81 and 86 have a general flange-like shape configuration when viewed in cross-section that is also some what L-shaped with the beads 100 and 101 forming feet of the flange shape that overlap and lock so as to prevent substantial radial movement of the arms 99 of the bone screw 89 relative to the closure plug base 85.

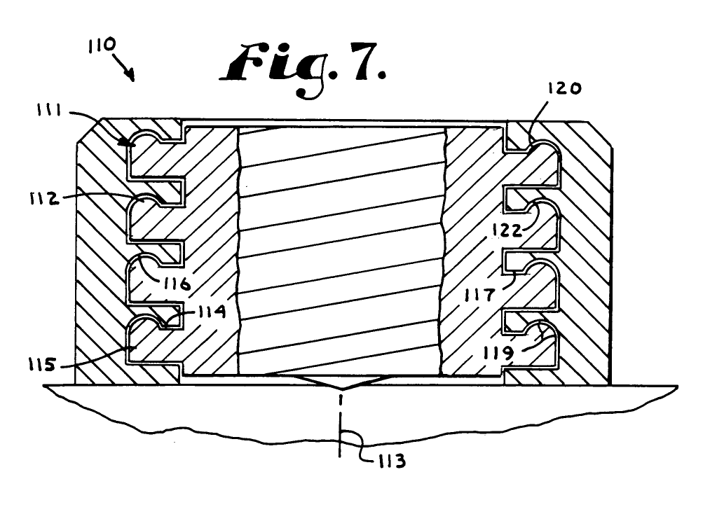

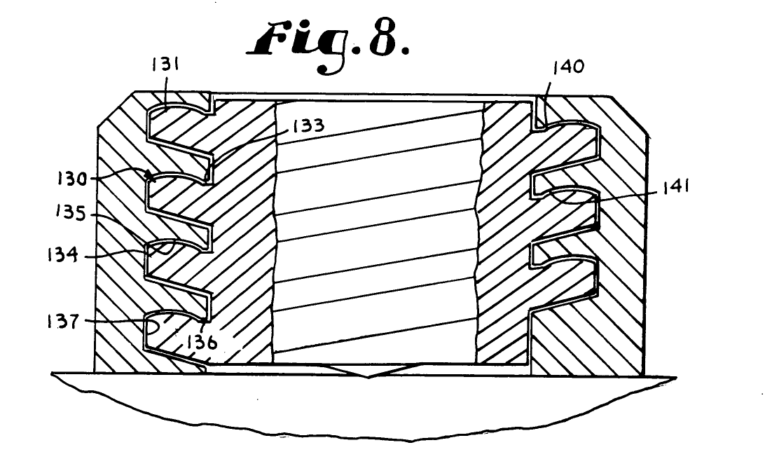

FIGS. 7 and 8 illustrate further variations of the axial shoulder interlocking structure 110 and 130 respectively in the form of a rounded axial bead interlocking form 111 shown in FIG. 7 and a shallow rounded axial bead interlocking form 131 in FIG. 8. The rounded axial bead interlocking form 111 includes a rounded bead 112 projecting in a direction parallel to an axis 113. The bead 112 is formed on a thrust surface 114 of an external interlocking form 115 and a rounded groove 116 is formed on a thrust surface 117 of an internal interlocking form 119. The bead 112 includes a splay resisting surface 120, while the groove 116 also includes a splay resisting surface 122.

In a similar manner, the shallow rounded axial bead interlocking form 130 includes a shallow rounded bead 131 formed on a thrust surface 133 of an external interlocking form 134 and a shallow rounded groove 135 formed on a thrust surface 136 of an internal interlocking form 137. The bead 131 includes a splay resisting surface 140, and the groove 135 includes a splay resisting surface 141. The surfaces 140 and 141 engage and abut to resist splaying or significant radial separation movement therebetween.

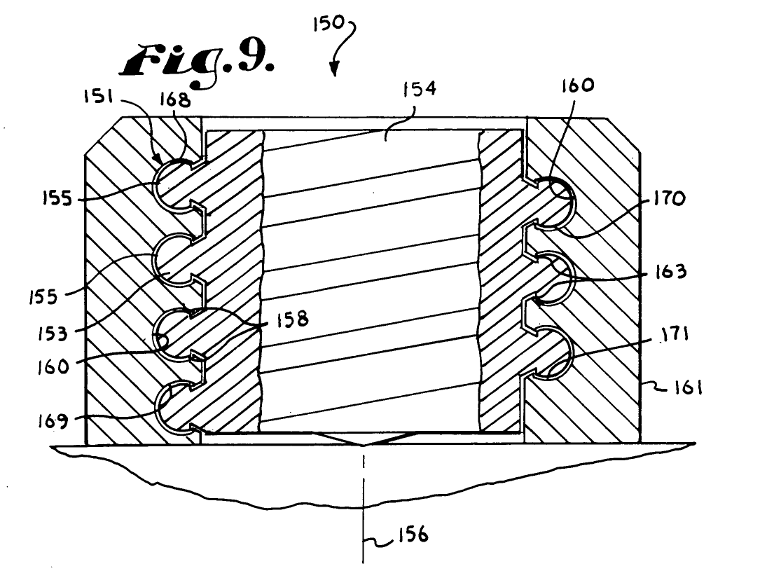

FIG. 9 illustrates a radial bead embodiment of an implant 150 having a guide and advancement structure 151. The structure 151 includes a rounded external and bead interlocking form 153 projecting radially from a base 154 and forming a crest 155. The bead interlocking form 153 has a pair of splay resisting surfaces 158 facing generally toward an axis 156 of rotation of the base 154. A complementary groove internal interlocking form 160 is part of a screw head 161. The head interlocking form 160 has a pair of splay resisting surfaces 163 facing generally away from the axis 156. The structure 151 has the splay resisting surfaces 158 and 163 on thrust surfaces 168 and 169 respectively of the interlocking forms 153 and 160, as well as on clearance surfaces 170 and 171 thereof. The illustrated radial bead interlocking form 150 is, in some ways, a double sided variation of the rounded axial bead interlocking form of an earlier embodiment.

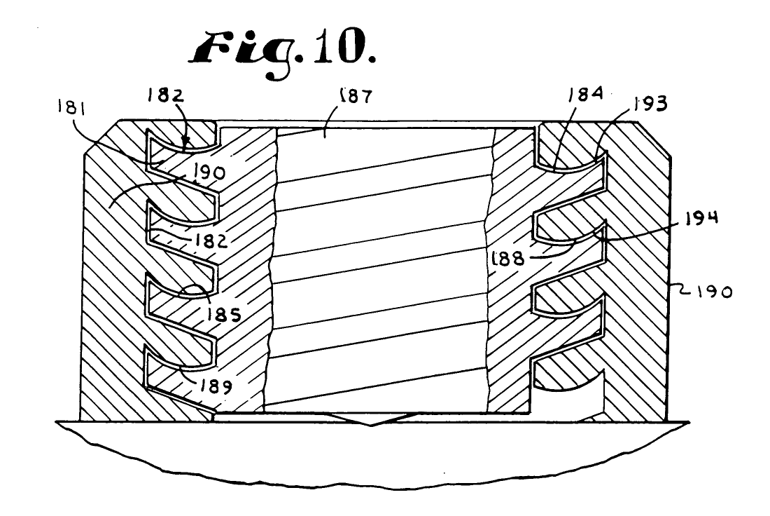

FIGS. 10 and 11 illustrate a scalloped, scooped or curvate surface embodiment structure 180 including a pair of compound interlocking forms 181 and 182 according to the present invention. The interlocking form 181 is scalloped and, in effect, an inversion of the shallow rounded bead interlocking form similar to that of an earlier embodiment. The interlocking form 182 includes a shallow groove 184 formed in a thrust surface 185 of the external interlocking form 181 of a base 187 and a shallow bead 188 formed on a thrust surface 189 of the interlocking form 182 of a screw head 190. The groove 184 has a splay resisting surface 193 which cooperates with a complementary splay resisting surface 194 of the bead 188.

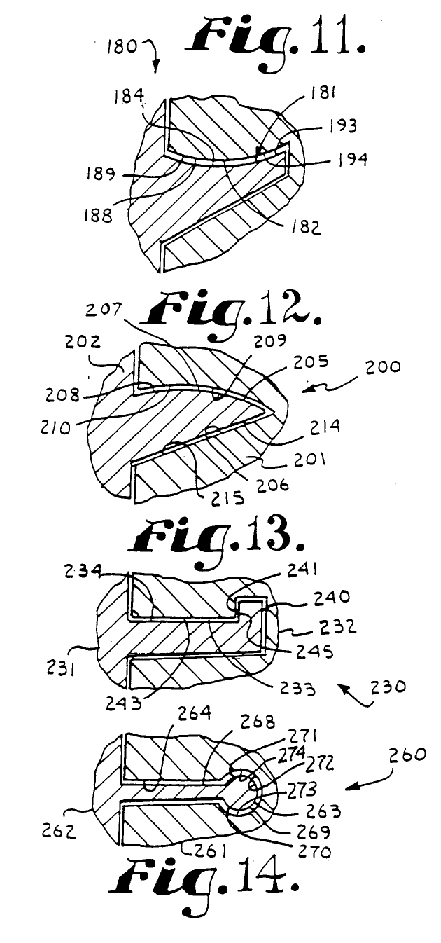

Illustrated in FIG. 12 is another guide and advancement structure 200 associated with a receiver member 201 and a closure member, such as a plug, 202 that is rotated into the receiver member 201. The structure 200 includes a first interlocking form 205 and a second interlocking form 206 attached to the closure member 202 and receiver member 201 respectively.

The first interlocking form 205 includes an arcuate upper surface 207 with a gripping or locking section 208. The second interlocking form 206 includes an arcuate lower surface 209 with a gripping or locking section 210. The interlocking forms 205 and 206 also have respective lower or leading surfaces 214 and 215 respectively that are sufficiently spaced to allow rotation about the axis thereof, but sufficiently close to be snug and not allow substantial movement of the forms 205 and 206 relative to each other in an axial direction without rotation.

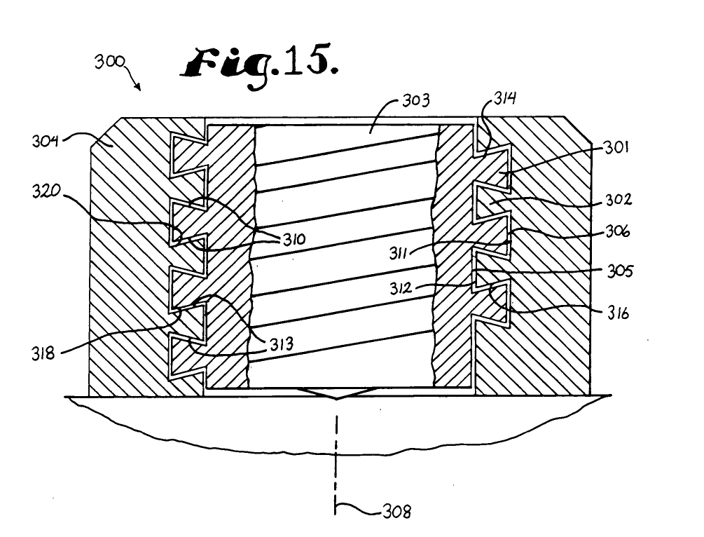

FIG. 13 shows an alternative flange shaped embodiment of a guide and advancement structure 230 in accordance with the invention. The structure 230 is mounted on a closure 231 and a receiver 232 so that interlocking forms 233 and 234, which are seen in cross-section, are helically mounted on the closure 231 and receiver 232 respectively.

The first interlocking form 233 is L or flange-shaped in cross-section with a vertically or axially extending foot portion 240 with a gripping surface 241. The second interlocking form 234 generally complements the first and is also L or flange shaped except that a foot 243 thereof is much wider than the foot portion 240. The foot 243 has a gripping or wraparound surface 245 that abuts the surface 241 during assembly and resist radial movement between the receiver 232 and the closure 231.

Shown in FIG. 14 is another embodiment of a guide and advancement structure 260 in accordance with the invention. The structure 260 is utilized with a receiver 261 and a closure or plug 262. The structure 260 has first and second interlocking forms 263 and 264. The first interlocking form has an elongate wall 268 with a circular bead 269 attached to an end thereof opposite the closure 262. The bead 269 has opposed gripping surfaces 270 and 271. The second interlocking form 264 is shaped to mate with an generally surround the first interlocking form 263 except sufficient clearance is provided to allow the closure 262 to be rotated and advanced into the receiver 263 by sliding tangentially, but not radially. The second interlocking form 264 has a circular cross-sectional channel 272 that receives the bead 269 and a pair of gripping surfaces 273 and 274 that engage and abut against the bead surfaces 270 and 271.

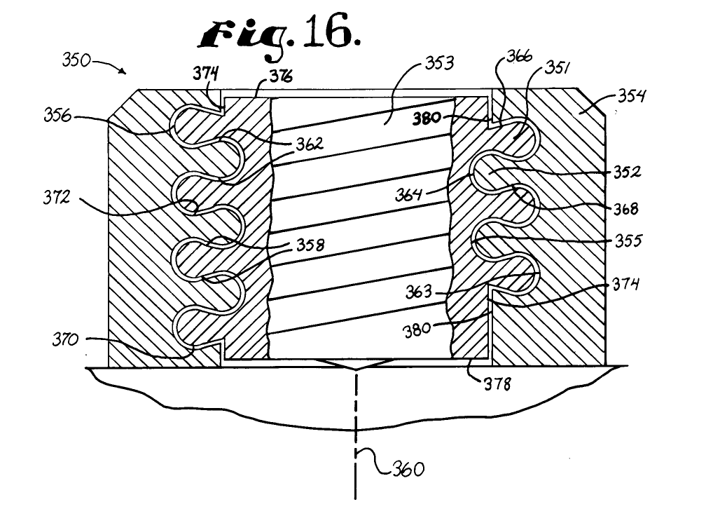

FIG. 15 illustrates a dove-tail or reverse wedge type embodiment of a guide and advancement structure generally 300 according to the invention that includes first and second substantially similarly shaped interlocking forms 301 and 302. Each of the interlocking forms 301 and 302 are in the form of a helically wound reverse wedge or dove-tail shape with leading and trailing surfaces diverging outwardly from one another in a direction from a root to a crest thereof. The structure 300 is utilized with a closure or plug 303 and a receiver 304. The form 301 is disposed on the plug 303 and projects outwardly radially from a root surface 305 to a crest surface 306. Both the root and crest surfaces 305 and 306 are disposed substantially parallel to an axis of rotation 308 of the plug 304. The interlocking form 301 has a pair of splay resisting surfaces 310 of substantially the same length, each linear in axial cross-section and each disposed at substantially the same angle with respect to the axis of rotation 308, but opening and extending in opposed directions, diverging outwardly from one another. Each of the splay resisting surfaces 310 generally faces toward the axis of rotation 308. Stated in another way, when looking at a cross-section taken in a plane passing through the axis 308, the form 301 has an axially aligned thickness along the cross-section that uniformly increases in both directions along the axis 308 as the structure 301 extends from the root 305 toward the crest 306.

The complimentary second interlocking form 302 is on the receiver 304. The interlocking form 302 projects inwardly radially from a root surface 311 to a crest surface 312. Both the root and crest surfaces 311 and 312 are disposed substantially parallel to the axis of rotation 308 when the forms 301 and 302 are engaged. The form 302 further includes a pair of splay resisting surfaces 313 of substantially the same length, each linear in axial cross-section and each disposed at substantially the same angle with respect to the root surface 311, but opening and extending in opposed directions, diverging outwardly from one another. The splay resisting surfaces 313 generally face away from the axis of rotation 308 when the forms 301 and 302 are engaged. Substantially similar to the form 301, the form 302 has a uniformly increasing axial thickness when viewed in cross-section along a plane passing through the axis 308 as the form extends inwardly radially, as illustrated in FIG. 15.

The structure 300 has the splay resisting surfaces 310 and 313 on thrust surfaces 314 and 316 respectively of the interlocking forms 301 and 302, as well as on respective clearance surfaces 318 and 320 thereof. The splay resisting surfaces 310 and 313 are sufficiently spaced to allow rotation of the plug closure 303 about the axis 308, but sufficiently close to be snug and not allow substantial movement of the forms 301 and 302 relative to each other in an axial direction without rotation. The illustrated dove-tail interlocking structure 300 is, in some ways, a non-arcuate double sided variation of the rounded axial bead interlocking form of an earlier embodiment.

FIG. 16 illustrates another radial bead type guide and advancement structure generally 350 according to the invention that may also be described as a double sided variation of the axial bead interlocking form of an earlier embodiment. The structure 350 includes first and second substantially uniform similarly shaped helically wound interlocking forms 351 and 352. Each of the interlocking forms 351 and 352 are of a jig-saw puzzle-like shape when viewed in axial cross-section, with leading and trailing surfaces being curvate and convex over a substantial portion thereof. The structure 350 is utilized with a closure or plug 353 and a receiver 354. The form 351 is disposed on the plug 353 and projects radially from a root 355 to a crest 356. The interlocking form 351 has a pair of curved substantially similarly shaped splay resisting surfaces 358 of substantially the same length, each surface 358 facing generally towards an axis of rotation 360 of the plug 353. When looking at a cross-section taken in a plane passing through the axis 360 as is illustrated in FIG. 16, the form 351 has an axially aligned thickness along such a cross-section that generally increases from the root 355 toward the crest 356 along a substantial portion of the structure 351. However, because of the jig-saw puzzle-like shape the form 351, the axially aligned thickness of the form uniformly decreases, then increases, and then decreases again in both axial directions as the form 351 extends radially outwardly to the crest 356. Thus, because of the rounded nature of the form 351, an axially aligned thickness near the crest 356 is smaller than an axially aligned thickness disposed, for example, midway between the root 355 and the crest 356. Also because of the jig-saw-like shape of the form 351, an axially aligned thickness near the root 355 is larger, for example, than another axially aligned thickness spaced at a slightly greater distance from the root 355.

The complimentary second interlocking form 352 is on the receiver 354. The interlocking form 352 has a pair of splay resisting surfaces 362 facing generally away from the axis of rotation 360 when the forms 351 and 352 are engaged. Also when engaged with the form 351, the form 352 generally projects inwardly radially toward the axis of rotation 360 from a root 363 to a crest 364. The form 352 has an axial thickness that varies substantially similar to, but also complimentary to the form 351, when viewed in cross-section along a plane passing through the axis 360 as illustrated in FIG. 16. The axially aligned thickness of the form 352 uniformly decreases, then increases, and then decreases again in both axial directions as the form 352 extends radially inwardly to the crest 364.

The structure 350 has the splay resisting surfaces 358 and 362 on thrust surfaces 366 and 368 respectively of the interlocking forms 351 and 352, as well as on respective clearance surfaces 370 and 372 thereof. The splay resisting surfaces 358 and 362 are sufficiently spaced to allow rotation of the plug closure 353 about the axis 360, but sufficiently close to be snug and not allow substantial movement of the forms 351 and 352 relative to each other in an axial direction without rotation.

The embodiment 350 also shows at least one overlapping root and crest configuration for the interlocking forms, which may be described as follows: The form 351 on the plug 353 includes a root 374 in addition to the root 355. The root 374 may be described as a first root, while the root 355 may be described as a second root, because the first root 374 is located at a leading end of the form 351. The root 374 is also at the trailing end of the form 351, with the second root 355 disposed between the roots 374. Stated in another way, as illustrated in FIG. 16, the first root 374 is disposed near a top surface 376 and a bottom surface 378 of the plug 353. The first root 374 cooperates with an inner cylindrical surface 380 of the receiver 354 and has a radius measured from the axis of rotation 360 that is larger than a radius of the second root 355, also measured from the axis of rotation 360. When the plug 353 is inserted in the receiver 354 and the form 351 engages the form 352, the crest 364 of the receiver 354 is disposed adjacent to the second root 355 and is also disposed inwardly radially from the root 374, closer to the axis of rotation 360 than the first root 374 of the closure or plug 353. Thus, the crest 364 is disposed radially between the axis of rotation 360 and the first root 374. Stated in another way, the crest 364 overlaps the first root 374 when cooperating with the second root 355.

It is foreseen in accordance with the invention that certain regions of the interlocking forms may be eased or removed to allow for easier use which still maintaining the primary objective of resisting radial movement between the closure plug and the opposed arms of the bone screw to prevent splaying of such arms.

It is also seen in accordance with the invention that the axial aligned extension or depression on the described interlocking forms could in some cases be multiple in nature or formed by an undulating pattern.

It is to be understood that while certain forms of the present invention have been illustrated and described herein, it is not to be limited to the specific forms or arrangement of parts described and shown.

What is claimed and desired to be secured by Letters Patent is as follows:

1. A closure for setting engagement with a structural member, the closure comprising:
(a) a substantially cylindrical body having an outer surface substantially uniform relative to a central closure axis; and
(b) a substantially continuous guide and advancement structure extending helically about said outer surface, said structure extending radially outwardly from a root to a crest thereof and having an axially aligned thickness along a cross-section taken in a plane passing through the axis, the thickness of a substantial portion of the structure increasing in a direction from the root toward the crest.

2. The closure of claim 1 wherein the guide and advancement structure has a first axially aligned thickness near the root, a second distinct axially aligned thickness near the crest and a third distinct axially aligned thickness between the first and second axially aligned thicknesses, the third axially aligned thickness being larger than the first axially aligned thickness and smaller than the second axially aligned thickness.

3. The closure of claim 1 wherein the guide and advancement structure has a first axially aligned thickness near the root, a second distinct axially aligned thickness near the crest and a third distinct axially aligned thickness disposed between the first and second axially aligned thicknesses, the third axially aligned thickness being smaller than both the first and second axially aligned thicknesses.

4. The closure of claim 3 wherein the second axially aligned thickness is smaller than the first axially aligned thickness.

5. The closure of claim 1 wherein the axially aligned thickness increases then decreases from the root to the crest.

6. The closure of claim 1 wherein the axially aligned thickness decreases then increases from the root to the crest.

7. The closure of claim 1 wherein the guide and advancement structure has a first linear leading surface and a second linear trailing surface, the first and second surfaces diverging outwardly from one another in a direction from the root to the crest.

8. The closure of claim 1 wherein the guide and advancement structure has a leading surface and a trailing surface, at least one of the leading and trailing surfaces being substantially convex.

9. The closure of claim 8 wherein both the leading surface and the trailing surface are substantially convex.

10. In a medical implant having a substantially cylindrical closure member with a helical guide and advancement structure thereon, the closure member having a longitudinal axis and an external surface, the improvement comprising:
a) a projection extending from the external surface, the projection having a uniform radial length measured between a root of the projection and a crest thereof, the projection having an axially aligned thickness along a cross-section taken in a plane passing through the axis, the axially aligned thickness of a substantial portion of the projection increasing along the radial length in a direction from the root toward the crest.

11. The improvement of claim 10 wherein the axially aligned thickness increases then decreases from the root to the crest.

12. The improvement of claim 10 wherein the axially aligned thickness decreases then increases from the root to the crest.

13. The improvement of claim 10 wherein the axially aligned thickness decreases then increases then decreases from the root to the crest.

14. The improvement of claim 10 wherein the medical implant is a polyaxial bone screw assembly and further comprising an elongate connecting member receivable in the bone screw assembly, the closure member causing the connecting member to bear directly against a top portion of a bone screw shank of the polyaxial bone screw assembly.

15. In a medical implant having a helical guide and advancement structure on a receiver, the receiver having a longitudinal axis and an internal surface, the improvement comprising:
a) a projection extending from the internal surface, the projection having a uniform radial length measured between a root of the projection and a crest thereof, the projection having an axially aligned thickness along a cross-section taken in a plane passing through the axis, the axially aligned thickness of a substantial portion of the projection increasing along the radial length in a direction from the root toward the crest.

16. The improvement of claim 15 wherein the axially aligned thickness increases then decreases from the root to the crest.

17. The improvement of claim 15 wherein the axially aligned thickness decreases then increases from the root to the crest.

18. The improvement of claim 15 wherein the axially aligned thickness decreases then increases then decreases from the root to the crest.

19. The improvement of claim 15 wherein the medical implant is a polyaxial bone screw assembly having a closure member with a guide and advancement structure thereon interlockable with the receiver guide and advancement structure and further comprising an elongate connecting member receivable in the receiver, the closure member causing the connecting member to bear directly against a top portion of a bone screw shank of the polyaxial bone screw assembly.

20. In a medical implant having a closure member, the closure member having a helical guide and advancement structure on an external surface thereof, the improvement comprising:
a) a projection extending from the external surface, the projection having a first linear leading surface and a second linear trailing surface, the first and second surfaces diverging outwardly from one another in a direction from a root to a crest of the guide and advancement structure.

21. The improvement of claim 20 wherein the guide and advancement structure is a first guide and advancement structure and the projection is a first projection and further comprising a receiver having a second projection extending from an internal surface thereof, the second projection having a third linear leading surface and a fourth linear trailing surface, the third and fourth surfaces diverging outwardly from one another and the first projection interlockable with the second projection.

22. The improvement of claim 21 further comprising an elongate connecting member receivable by the receiver and lockable within the receiver by the closure member, the closure member causing the elongate member to directly contact and bear against a top end of a bone screw shank of a polyaxial bone screw.

23. In a medical implant having a closure member, the improvement wherein the closure member has a helical guide and advancement structure having a projection for interlocking engagement with a bone screw receiver, the projection having a leading surface and a trailing surface, at least one of the leading and trailing surfaces being substantially convex.

24. The improvement of claim 23 wherein both the projection leading surface and the projection trailing surface are substantially convex.

25. The improvement of claim 23 wherein the medical implant is a polyaxial bone screw assembly having a receiver swivelably connected to a bone screw shank and further comprising an elongate connecting member receivable by the receiver and lockable within the receiver by the closure member, the closure member causing the elongate member to directly contact and bear against a top end of the bone screw shank.

26. In a medical implant having a closure member with an axis of rotation and a first helical guide and advancement structure and a receiver having a second helical guide and advancement structure mateable with the first helical guide and advancement structure, the improvement comprising:
a) a first projection on the first helical guide and advancement structure, the first helical guide and advancement structure having a first root at a leading end thereof and a second root; and
b) a second projection on the second helical guide and advancement structure, the first projection interlockable with the second projection, the second helical guide and advancement structure having a crest, and wherein when the second projection is interlocked with the first projection, the crest is disposed adjacent to the second root and radially between the axis of rotation and the first root.

27. In a closure apparatus having a closure member with a first helical guide and advancement structure having a leading surface and a trailing surface and a receiver having a second helical guide and advancement structure rotatably mateable with the first helical guide and advancement structure, the improvement comprising:
a) a first interlocking form on the first guide and advancement structure; and
b) a second interlocking form on the second guide and advancement structure, the first and second forms sized and shaped to interlock with one another and resist disengagement by a radially outwardly pulling force both in the presence of and in the absence of a loading force on the trailing surface.
